(19) United States
(12) Patent Application Publication
(10) Pub. No.: US 2006/0283656 A1
(43) Pub. Date: Dec. 21, 2006

(54) Bell side structure for stethoscope head

(76) Inventor: Tung-Yi Lin, Banchiau City (TW)

Correspondence Address: Tung-Yi Lin, P.O. Box No. 6-57, Junghe, Taipei, 235, TW

(21) Appl. No.: 11/154,549

(22) Filed: Jun. 17, 2005

(51) Int. Cl. A61B 7/02

(52) U.S. Cl. 181/131

(57) ABSTRACT

A bell side structure for a stethoscope head of the present invention is related to a design for a stethoscope head, and more particularly to a bell side structure fabricated from a metallic diaphragm. The metallic diaphragm includes a metallic membrane, and superior conductibility of metallic material is used to increase conduction efficiency of sound waves by the bell side, thereby increasing diagnostic effectiveness of the stethoscope head.

### BACKGROUND OF THE INVENTION

(a) Field of the Invention

The present invention relates to a design for a stethoscope head, and more particularly to a bell side structure fabricated from a metallic diaphragm made from metallic material, and uses the superior conductibility of metallic material to increase conduction efficiency of sound waves by the bell side.

(b) Description of the Prior Art

In general, in order to effectively diagnose the health status of the organs of a patient, a medical staff member places a stethoscope on the skin surface at the position of each of the major organs, such as the heart, the stomach, the lungs, the bronchial tube and the various joints, and the sound waves generated by oscillations of the inner organs are transmitted to the ear of the medical staff member, who is then able to diagnose from the contractions or oscillation patterns of each of the organs whether they are in good health or not.

The stethoscopes currently used by medical staff member can be roughly divided into two types, including a single-sided stethoscope and a dual-sided stethoscope, both of which have a bell side (or drumhead) disposed on an end portion of the stethoscope head. The bell side is placed on the skin surface corresponding to the position of an organ to sense the oscillations or pulsations of the organ of the patient, thereby causing the bell side to vibrate and generate sound waves, which enable the medical staff member to positively diagnose the health status of the organ from the oscillations or pulsations.

Referring to FIGS. 1 and 2, which illustrate a conventional dual-sided stethoscope head 10, consisting of a sound guide connector (20), a sound guide channel (30) that longitudinally penetrates the sound guide connector (20), and a high frequency pickup (50) and a low frequency pickup (60) disposed on upper and lower ends of the sound guide connector (20) respectively. The sound guide channel (30) connects to a Y-shaped tube (40), and protruding bell sides (or drumheads) (90) are respectively configured on end portions of the high frequency pickup (50) and the low frequency pickup (60). The bell sides (90) are primarily fabricated from plastic material, and assume laminate forms.

The protruding bell sides (90) respectively configured on the high frequency pickup (50) and the low frequency pickup (60) of the upper and lower ends of the dual-sided stethoscope head (10) are placed on the skin of a patient to sense the vibration effect of the oscillations generated by the oscillations and pulsations of the organs of the patient, whereupon the sound waves generated within the high frequency pickup (50) or the low frequency pickup (60) are transmitted to the Y-shaped tube (40) through the sound guide connector (20) and the sound guide channel (30), and thereon transmitted to the ear of the medical staff member, who is then able to clearly hear whether the oscillations or pulsations of the organ are normal or not, and thus positively diagnose the illness of the patient.

However, the traditional bell side (90) is fabricated from plastic material, and thus frequency of the sound waves transmitted by the oscillating phenomenon is affected because of the unideal transmission efficiency of sound waves of plastic material, which causes complications for the medical staff member when diagnosing the illness of the patient.

### SUMMARY OF THE INVENTION

Hence, in light of the urgency to resolve the aforementioned long-standing problems, the inventor of the present invention, having accumulated years of experience in research, design and manufacture of related arts, attentively and circumspectly carried out extensive study and exploration, and, after repeated experimentation and testing, ultimately designed a bell side structure for a stethoscope head of the present invention.

Accordingly, a primary objective of the present invention is to provide the bell side structure for a stethoscope head that primarily uses a metallic diaphragm made from metallic material and superior conductibility of the metallic material to increase conduction efficiency of sound waves by the bell side, thereby increasing diagnostic effectiveness of the stethoscope head.

Another objective of the present invention is to further superimpose a protective membrane on one side or two sides of the metallic diaphragm, thereby increasing structural strength and maintaining cleanliness of the metallic diaphragm of the metallic diaphragm.

To enable a further understanding of said objectives and the technological methods of the invention herein, brief description of the drawings is provided below followed by detailed description of the preferred embodiments.

### BRIEF DESCRIPTION OF THE DRAWINGS

FIG. 1 shows an exploded schematic view of a dual stethoscope head of prior art.

FIG. 2 shows a schematic cross-sectional view of the dual stethoscope head of prior art.

FIG. 3 shows a schematic elevational view according to the present invention.

FIG. 4 shows an exploded schematic view according to the present invention.

FIG. 5 shows a schematic cross-sectional view according to the present invention.

FIG. 6 shows a schematic cross-sectional view of another embodiment according to the present invention.

FIG. 7 shows a schematic cross-sectional view of yet another embodiment according to the present invention.

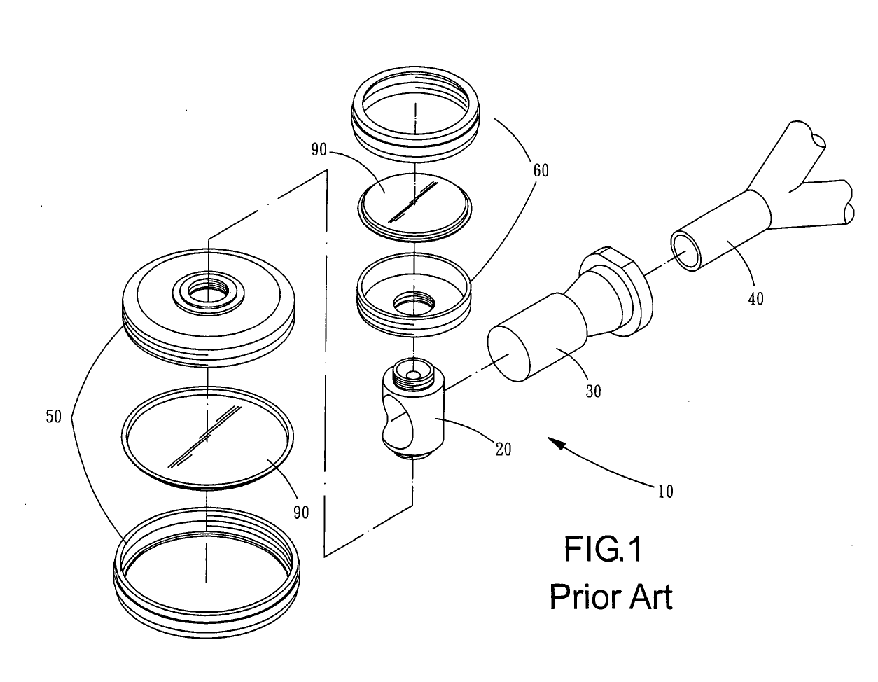

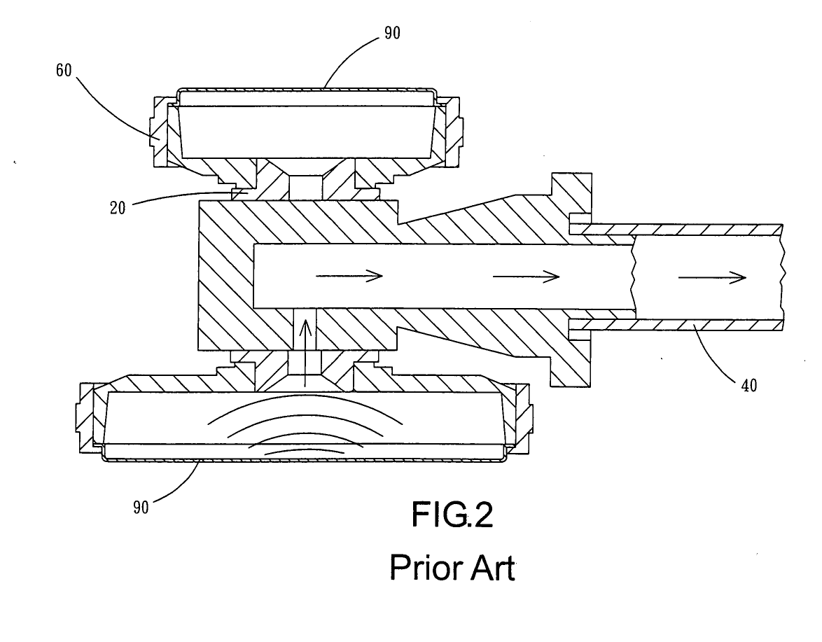

### DETAILED DESCRIPTION OF THE PREFERRED EMBODIMENTS

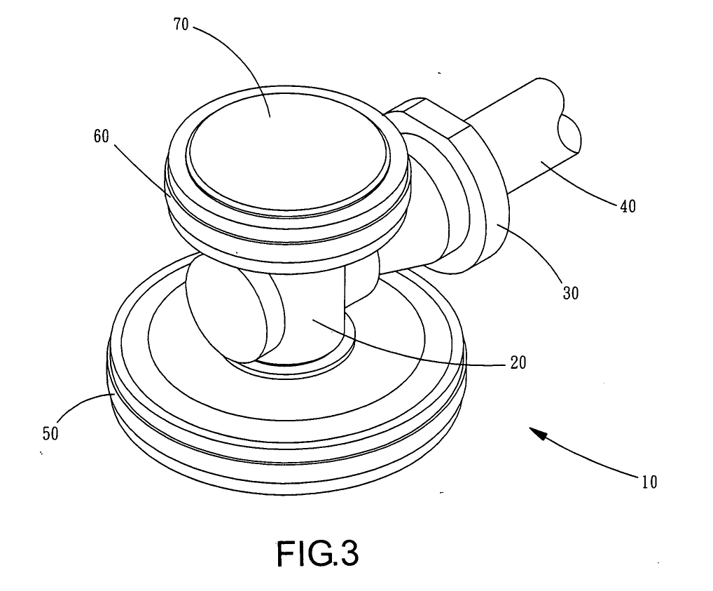

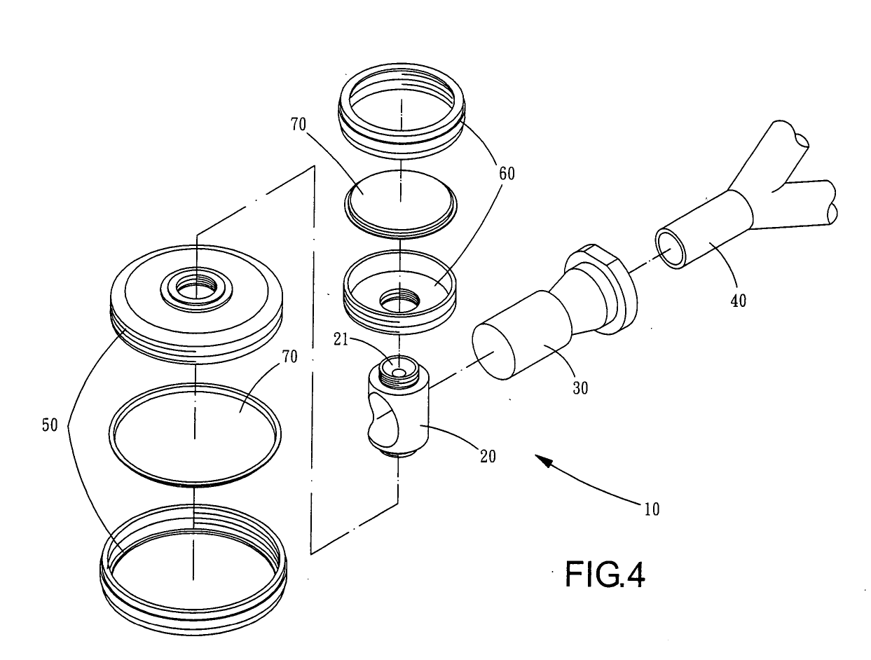

Referring to FIGS. 3 and 4, which illustrate a dual-sided stethoscope head embodiment of a bell side structure for a stethoscope head of the present invention, although the bell side structure for a stethoscope head is also suitable for application in a single-sided stethoscope head. The dual-sided stethoscope head (10) comprises:

a sound guide connector (20), two ends of which have and a sound chamber (21) disposed thereon;

a sound guide channel (30) longitudinally penetrating the sound guide connector (20), which affords a channel to the sound chambers (21), and is used to connect to a Y-shaped tube (40); and

a high frequency pickup (50) and a low frequency pickup (60) disposed on an upper end and a lower end of the sound guide connector (20) respectively, a protruding bell side (or drumhead) is disposed on an end portion of the high frequency pickup (50) and the low frequency pickup (60), and the bell sides are respectively positioned exactly on outer sides of the sound chambers (21).

The present invention is characterized in that the bell sides are primarily fabricated from metallic material harmless to the human body, such as gold, silver, copper, aluminum, and so on.

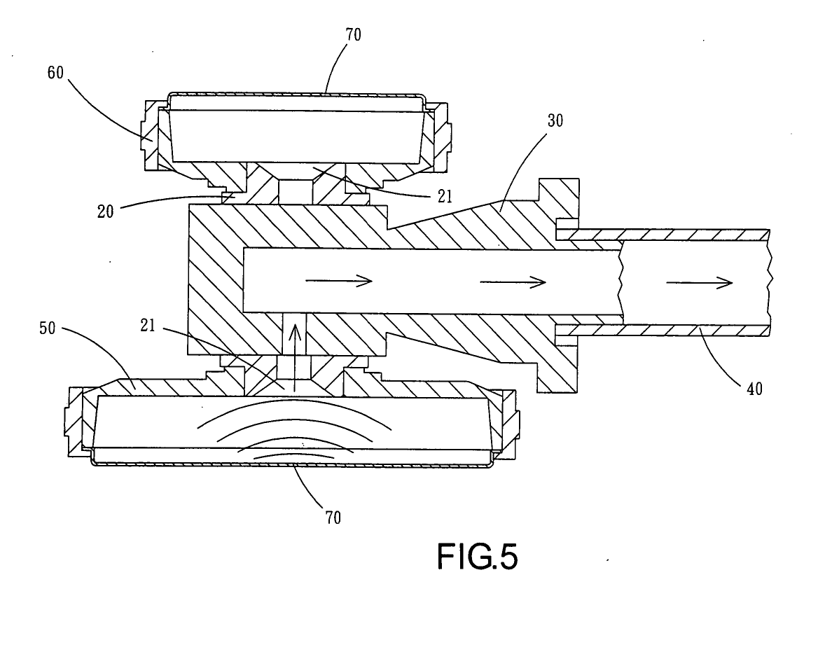

Referring to FIG. 5, when the high frequency pickup (50) or the low frequency pickup (60) of the stethoscope head (10) is placed on the skin corresponding to an organ of a patient, contact between the bell side and the skin is used to sense oscillations or pulsations of the organ, which cause the bell side to oscillate along with vibrations of the skin surface, thereby generating sound waves in one of the sound chambers (21) of the sound guide connector (20).

Because each of the bell sides of the present invention is fabricated from a metallic diaphragm (70) that is made from metallic material, thus, the superior conductibility of metallic material can be used to enable the bell sides to generate a clearer and truer vibration frequency, which thus transmit steady sound waves to the ear drum of the medical staff member, who is then able to make a more accurate diagnosis of the illness of a patient.

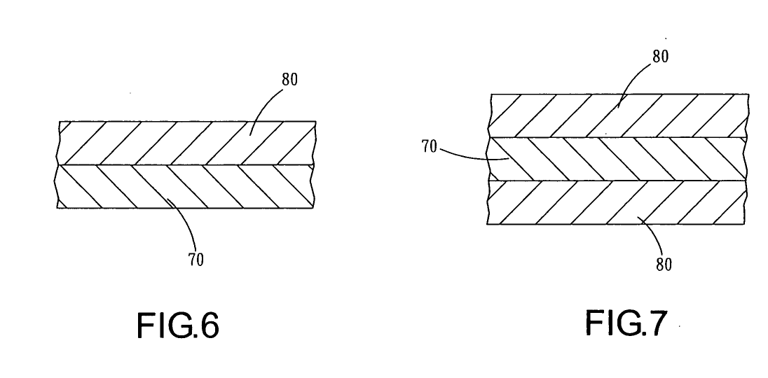

Furthermore, referring to FIG. 6, in order to increase structural strength of the metallic diaphragm (70), a protective membrane (80) is superimposed on any one side of the metallic diaphragm (70). Moreover, referring to FIG. 7, the protective membrane (80) can be superimposed on two sides of the metallic diaphragm (70), thereby increasing structural strength and maintaining cleanliness of the metallic diaphragm (70).

Because of the many structures and forms of a stethoscope head, thus, shape of the metallic diaphragm (70) of the present invention can be varied according to structure and form of the stethoscope head, for instance, the metallic diaphragm (70) can be made to assume an inverted U-shape, a planar form, and so on.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A dual-sided structure for a stethoscope head comprising metallic diaphragms made from metallic material disposed on end portions of pickups; when one of the metallic diaphragms is placed on the skin surface of a patient, oscillations or pulsations of the organ cause the metallic diaphragm to vibrate, thereby generating sound waves within the pickups.

2. The dual-sided structure for a stethoscope head according to claim 1, wherein a protective member is further superimposed on one side of the metallic diaphragm, thereby increasing structural strength and maintaining cleanliness of the metallic diaphragm.

3. The dual-sided structure for a stethoscope head according to claim 1, wherein the protective member is further superimposed on each of two sides of the metallic diaphragm, thereby increasing structural strength of the metallic diaphragm.
